(19) United States
(12) Patent Application Publication
(10) Pub. No.: US 2009/0018551 A1
(43) Pub. Date: Jan. 15, 2009

(54) Back hair removal system

(76) Inventor: Warren B. Hampton, Temple Terrace, FL (US)

Correspondence Address: Warren B. Hampton, 520 Royal Greens Dr., Temple Terrace, FL, 33617, US

(21) Appl. No.: 12/217,819

(22) Filed: Jul. 9, 2008

Related U.S. Application Data
(60) Provisional application No. 60/959,282, filed on Jul. 13, 2007.

(51) Int. Cl. A61B 17/50

(52) U.S. Cl. 606/133

(57) ABSTRACT

A method and apparatus for removal of back hair involving the delivery of a depilatory agent by means of wearing a garment, cloth or article of clothing.

### CROSS-REFERENCE TO RELATED APPLICATIONS

This application claims the benefit of provisional patent application Ser. No. 60/959,282, filed Jul. 13, 2007 by the present inventor.

### FEDERALLY SPONSORED RESEARCH

Not Applicable

### SEQUENCE LISTING OR PROGRAM

Not Applicable

### BACKGROUND OF THE INVENTION

This application generally relates to hair removal, particularly to the method of removing back hair.

It is common practice among most of the world's population to remove hair in order to improve personal appearance. Females are especially desirous of removing unwanted hair on the face, underarms, legs and other anatomical parts that may be exposed because of current fashions such as bikini bathing suits and the like. Males are also concerned with removal of unwanted hair, including facial, armpit, chest and back hair.

Back hair occurs in males at a greater frequency and severity than females. A particular impediment to removal of back hair for the male is his relative inability to successfully accomplish the task alone, without the assistance of a friend or relative, or without the expense of hiring a professional at a salon or spa. To many men, unwanted back hair is an out-of-reach nuisance.

A viable solution for the do-it-yourself treatment/removal of unwanted back hair has been that represented by a razor attached to a straight or flexible extension rod. Devices of this sort are also known as “reaching razors” and are reflected in the general embodiments of U.S. Pat. No. 5,167,069, Dec. 1, 2002 to Kathleen Quinn, U.S. Pat. No. 6,266,888, Jul. 31, 2001 to Thomas E. Zowaski, U.S. Pat. No. 5,911,480, Jun. 15, 1999 to Danny K. Morgan, and U.S. Pat. No. 6,189,222, Feb. 20, 2001 to Calvin M. Doyle. While these may be effective in removing unwanted back hair, the user is still challenged by an awkward means to address and remove all unwanted back hairs on his back, all the while running the risk of cutting any back skin or any protrusion therein, such as a pimple, acne, wart and/or mole. Reaching over one's shoulder with a razor attached to the end of a rod to shave one's back has been a risky proposition.

Effectively utilizing a depilatory agent for the removal of back hair by the do-it-yourself subject has been relatively unheard of since there hasn't been an effective means by which to apply the depilatory agent without the assistance of a second person.

### SUMMARY OF THE INVENTION

Briefly, and in general terms, the present invention provides a new and improved hair removal system for delivering a depilatory agent onto a skin's surface area where back hair removal is desired, without the assistance of a second party.

Basically, the present invention is a system comprised of the methods and means for applying a suitable chemical depilatory agent to the skin surface of the back of one's torso where hair is to be removed, without any human assistance, i.e. do-it-yourself.

In a presently preferred embodiment, by way of example and not necessarily by way of limitation, an article of clothing, preferably a t-shirt or vest and a chemical depilatory are utilized for enhanced efficacy.

The inside of the shirt's or vest's back panel is given an application of a chemical depilatory agent, either by the manufacturer or by the user. The porous cloth will hold the depilatory agent for transfer. The user then wears the shirt or vest containing the depilatory agent. By doing so, the depilatory agent is delivered to the user's skin surface area containing the back hair to be treated. The back panel acts as the mechanism and means for delivering the chemical depilatory agent to the skin surface of the user's back torso containing the unwanted back hair.

After a predetermined amount of time, the user removes the shirt or vest and rinses his back. While rinsing, the user may use the shirt or vest to wipe or detach any remaining back hair through a back and forth motion utilizing both hands, much like drying one's back with a towel. The shirt or vest is then discarded as any remaining depilatory agent would damage or destroy it due to its causticity.

Hence, the new and improved back hair removal method and apparatus of the present invention enable more convenient, reliable, comfortable, convenient, and/or economical, do-it-yourself back hair removal.

These and other objects and advantages of the invention will become more readily apparent from the following more-detailed description of the invention, when taken in conjunction with the accompanying drawings of illustrative embodiments.

### DRAWINGS—FIGURES

FIG. 1 shows the front side of the apparatus with a back panel containing a chemical depilatory agent.

FIG. 2 shows a rear angle of the apparatus in use.

### DRAWINGS—REFERENCE NUMERALS

10 sleeve

20 collar

30 front of apparatus

40 inside of back panel containing chemical depilatory agent

50 back of apparatus

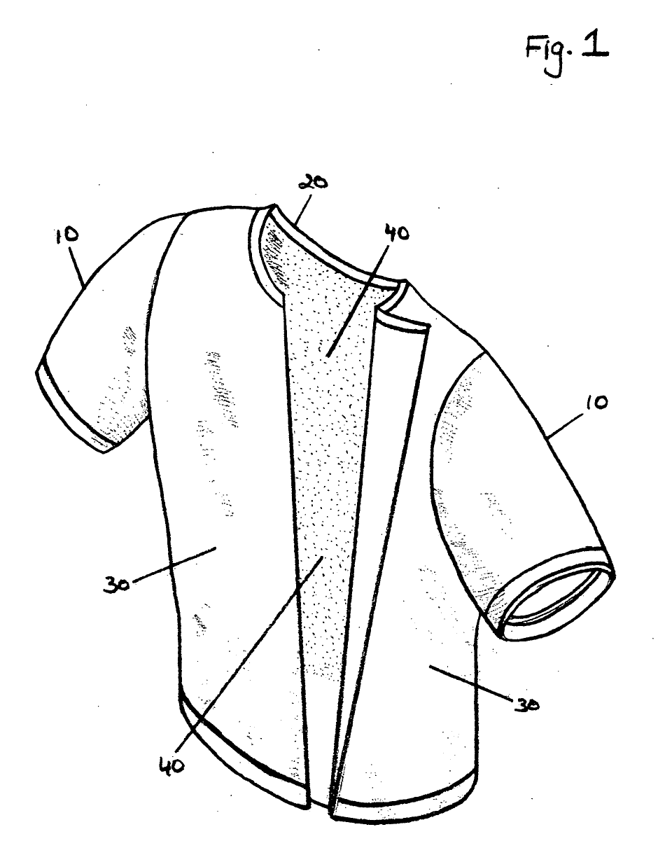

### DETAILED DESCRIPTION—FIG. 1—FIRST EMBODIMENT

One embodiment of the apparatus is illustrated in FIG. 1. The apparatus is comprised of a garment with two sleeves 10, a collar 20, and a back panel 40 that contains a chemical depilatory agent to be delivered to the targeted skin surface with back hair.

In the preferred embodiment, the apparatus would be comprised of a t-shirt (with an incision down its center, much like a regular button-down shirt or vest) or a shirt or vest permitting the user to put the apparatus on with ease, and with a back panel comprised of a cloth or porous material. The inside of the back panel 40 would contain most, if not all, of the applied chemical depilatory agent.

The apparatus's sleeves 10 and collar 20 ensure that the apparatus can be worn by a human subject of a comparable size.

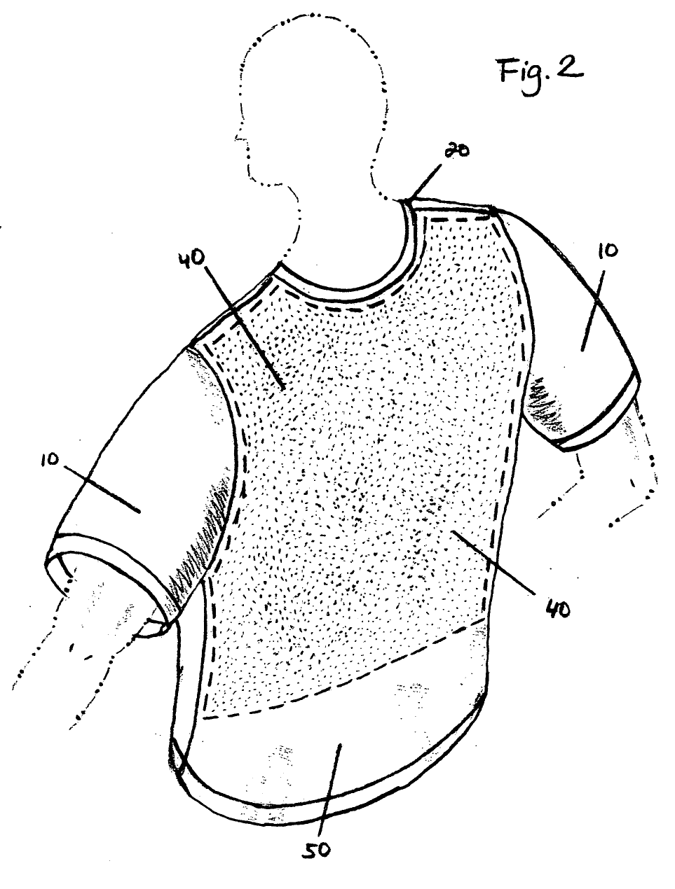

### OPERATION—FIG. 2

The manner of using the apparatus is similar to putting on and wearing a shirt, for a predetermined amount of time. The typical user would be a male wishing to treat unwanted back hair. Having the apparatus in a ready state, with the chemical depilatory agent applied onto the inside of the back panel 40, the user would put on the apparatus and wear said apparatus for a predetermined amount of time (typically an amount suggested by manufacturers of chemical depilators).

While wearing the apparatus, the chemical depilatory agent on the inside of the back panel 40 would come into contact with the skin surface containing unwanted back hair. Said contact could be enhanced via the human subject pulling tautly on the front of the apparatus 30.

After said predetermined amount of time, the user would remove the shirt and rinse the treated skin surface under running water, preferably in a shower. While rinsing, the human subject may use the apparatus to wipe off any remaining back hairs.

### CONCLUSION, RAMIFICATIONS, AND SCOPE

Accordingly, the reader will see that this new and improved back hair removal method and apparatus of the present invention enable more convenient, reliable, comfortable, and/or economical, do-it-yourself back hair removal.

It will be apparent from the foregoing that, while particular forms of the invention have been illustrated and described, various modifications can be made without departing from the spirit and scope of the invention. For example, the apparatus may be comprised of a towel, sheet, shawl, cloth, etc.

Thus the scope of the embodiments should be determined by the appended claims and their legal equivalents, rather than by the examples given.

What is claimed is:

1. An apparatus for the removal of back hair, comprising:
a. a wearable material suitable for the application of a chemical depilatory agent, having a chemical depilatory agent.

2. The apparatus in claim 1, wherein:
a. said wearable material is comprised of a garment, cloth, or article of clothing suitable for the upper torso.

3. The apparatus in claim 1, wherein:
a. said chemical depilatory agent is suitable for use on human subjects.

4. A method for removing back hair, comprising:
a. providing a wearable material suitable for the application of chemical depilatory agent,
b. providing a chemical depilatory agent,
c. providing said wearable material containing said depilatory agent,
d. wearing said wearable material containing said depilatory agent for a predetermined amount of time whereby said wearable material containing said chemical depilatory agent will come into contact with skin containing back hair and deliver said chemical depilatory agent to said skin and said back hair.

5. The method of removing back hair as set forth in claim 4, wherein:
a. said wearable material having a chemical depilatory agent is removed after predetermined amount of time.

6. The method of removing back hair as set forth in claim 4, wherein:
a. said wearable material having a chemical depilatory agent is used to wipe away any remaining back hairs.
